(19) United States
(12) Patent Application Publication
(10) Pub. No.: US 2018/0016230 A1
(43) Pub. Date: Jan. 18, 2018

(54) Salts of Prostaglandin Analog Intermediates

(71) Applicant: Apotex Inc., Toronto (CA)

(72) Inventors: Yajun Zhao, Brantford (CA); Yonggang Li, Shanghai City (CN); Uma Kotipalli, Brantford (CA); Sammy Chris Duncan, Brantford (CA); Honghai Lv, Tianjin City (CN); Kangying Li, Tianjin City (CN)

(21) Appl. No.: 15/534,183

(22) PCT Filed: Dec. 10, 2015
(86) PCT No.: PCT/CA2015/000597

§ 371 Date: Jun. 8, 2017

Related U.S. Application Data
(60) Provisional application No. 62/090,035, filed on Dec. 10, 2014.

(51) Int. Cl. C07C 405/00; C07F 7/18; C07C 211/38

(52) U.S. Cl. CPC C07C 405/00; C07C 211/38; C07F 7/1856; C07C 2603/74; C07C 2601/08; C07B 2200/13

(57) ABSTRACT

The present invention relates to crystalline 1-adamantanamine salts, and polymorphic forms thereof, of prostaglandin analog intermediates of formula 3a, 4a and 6a, useful in the preparation of Tafluprost and Lubiprostone and processes for their preparation. The process includes combining 1-adamantanamine, water, an organic solvent, and a compound of Formula 3 or 6, thereby obtaining a suspension. The process also includes isolating the solid salt of Formula 3a or 6a from the suspension.

### TECHNICAL FIELD

The present invention relates to intermediates useful in the preparation of prostaglandin analogs, in particular, to salts thereof.

### BACKGROUND

Lubiprostone (1) is a chloride channel activator and analog of Prostaglandin E1. It is marketed in the United States as AMITIZA™ and is indicated for the treatment of chronic idiopathic constipation in adults.

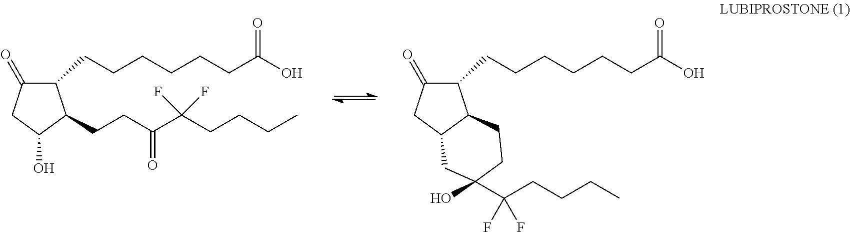 [SMILES: CCCCC(F)(F)C(=O)CC[C@H]1[C@H](O)CC(=O)[C@@H]1CCCCCCC(=O)O ; CCCCC(F)(F)[C@@]1(O)CC[C@H]2[C@@H](CC(=O)[C@@H]2CCCCCCC(=O)O)C1]

Tafluprost (2) is a fluorinated analog of prostaglandin F2alpha. It is marketed in the United States as ZIOPTAN™ and is indicated for reducing elevated intraocular pressure in patients with open-angle glaucoma or ocular hypertension.

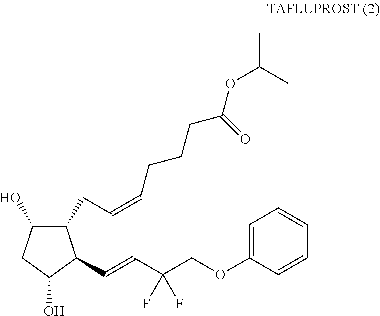 [SMILES: CC(C)OC(=O)CCC/C=C\C[C@@H]1[C@@H](/C=C/C(F)(F)COc2ccccc2)[C@H](O)C[C@@H]1O]

EP 0 850 926 A2 discloses a fluorine-containing prostaglandin derivative of the formula (A) or a salt thereof, and a medicine containing it, particularly, as a preventive or therapeutic medicine for an eye disease:

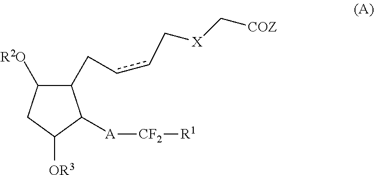

wherein A is a vinylene group or the like, R1 is an aryloxyalkyl group or the like, R2 and R3 are hydrogen atoms or the like, and Z is OR4, therein OR4 is a hydrogen atom or an alkyl group or the like.

Matsumura, Y. et al. in Tet. Lett. 2004, 45, 1527 discloses novel 15-deoxy-15,15-difluoro-prostaglandin(PG)F2alpha derivative AFP-168 synthesized from the Corey aldehyde in six steps. A key aspect of this route is difluorination of an enone and a stereoselective Wittig reaction. The compound shows high affinity to the FP receptor and potent activities for an anti-glaucoma agent.

U.S. Pat. No. 8,513,441 B2 discloses fused cyclopentane-4-substituted-3,5-dioxalane lactone compounds useful as intermediates in the synthesis of prostaglandin analogs. The compounds have the formula B:

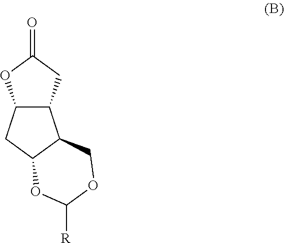

wherein R represents an aryl group such as p-methoxyphenyl. This compound can be reacted with a lower alkyl aluminum compound to open the dioxalane ring and reduce the lactone to lactol, without over-reducing to diol. The resulting compound can be functionalized to insert chemical side groups of target prostaglandins, adding the required alpha-side chain and then the required omega-side chain sequentially and independently of each other. The compounds and process are particularly suitable for preparing Lubiprostone.

WO 2010/096123 A2 is directed to novel amino acid prostaglandin salts and methods of making and using them.

WO 2010/083597 A1 provides processes for preparing Lubiprostone and intermediates thereof. Also provided are compounds, including intermediates for preparing Lubiprostone as well as compositions comprising Lubiprostone and other compounds, including intermediates for preparing Lubiprostone and other compounds.

CN 102101835 A discloses a prostaglandin derivative and a preparation method of a prostaglandin derivative intermediate. The intermediate is a compound shown as a formula (C),

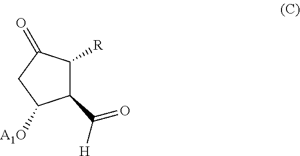

wherein A1 is a protecting group of hydrogen or acrinyl; R is —R1-Q; R1 is saturated or unsaturated bivalent low-grade or medium-grade aliphatic hydrocarbon which is unsubstituted or substituted by halogen, low-grade alkyl, hydroxyl, oxo radical, aryl or heterocyclic radical; at least one carbon atom in the aliphatic hydrocarbon is selectively substituted by oxygen, nitrogen or sulfur; and Q is —CH₃, —COCH3, —OH, —COOH or other functional group derivatives. The invention also discloses preparation methods of the intermediate and the prostaglandin derivative.

CN 102558009 A discloses a preparation method of a prostaglandin derivative. The method comprises the following steps: protecting corresponding hydroxyl in a prostaglandin intermediate body with alkoxy benzyl; and performing a hydrogenation reaction under a normal pressure to prepare a corresponding prostaglandin derivative. According to the method, the reaction condition is reduced, and the yield is improved. The method is more suitable for industrial production.

CN 103058907 A discloses a novel method for preparing a Lubiprostone midbody as shown in the formula J. The method comprises the following steps: (1) a compound as shown in the formula D reacts with tert-butyldimethylsilyl chloride to selectively protect a primary hydroxyl group, thereby obtaining a compound shown in the formula E; (2) a protecting group is applied to the compound E under the action of a catalyst, thereby obtaining a compound shown in the formula F; (3) after the compound F is reduced through diisobutylaluminium hydride, a Wittig reaction is carried out on the compound F, thereby obtaining carboxylic acid shown in the formula G; (4) the compound G is protected in an acetonitrile solvent through a protecting group, thereby obtaining a compound shown in the formula H; (5) the compound H is treated by using the tert-butyldimethylsilane for removing the protecting group, thereby obtaining a compound shown in the formula I; and (6) the compound I is oxidized by an oxidant and then reacts with a compound shown in the formula K, thereby obtaining the higher-purity compound shown in the formula J,

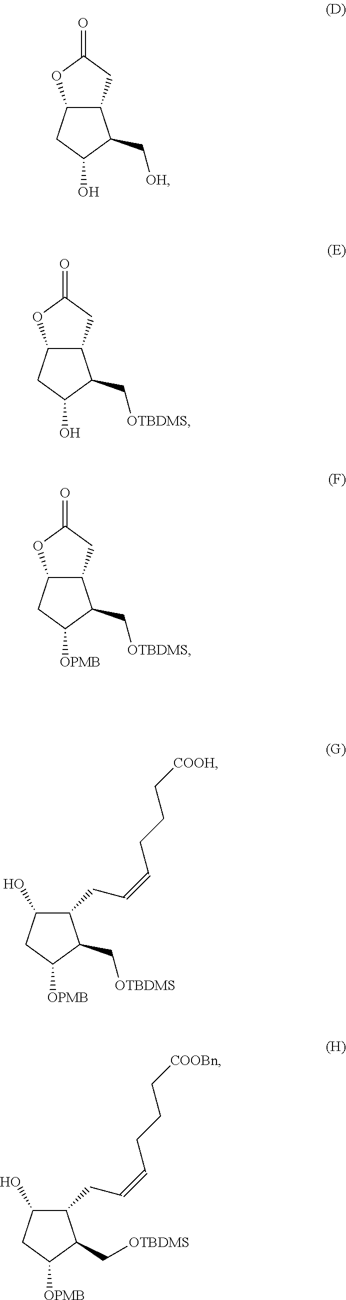

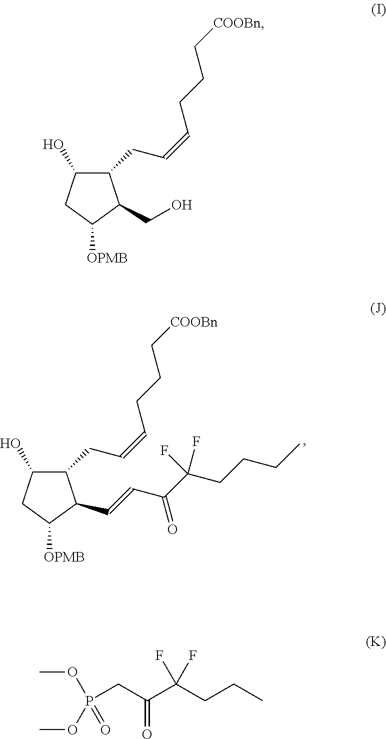

wherein TBDMS is tert-butyldimethylsilyl group; and PMB is a 4-methoxybenzyl.

WO 2013/118058 A1 relates to amine salts of prostaglandin analogs and their uses for the preparation of substantially pure prostaglandin analogs. Specific embodiments relate to amine salts of Tafluprost and their uses for the preparation of substantially pure Tafluprost.

### SUMMARY

The present invention is related, at least in part, to crystalline 1-adamantanamine salts and polymorphic forms thereof, of intermediates useful in the production of prostaglandin derivatives, including Lubiprostone and Tafluprost, and to the use thereof in the preparation of Lubiprostone and Tafluprost.

Illustrative embodiments of the present invention provide a crystalline salt of a prostaglandin analog intermediate having a formula selected from the group consisting of:

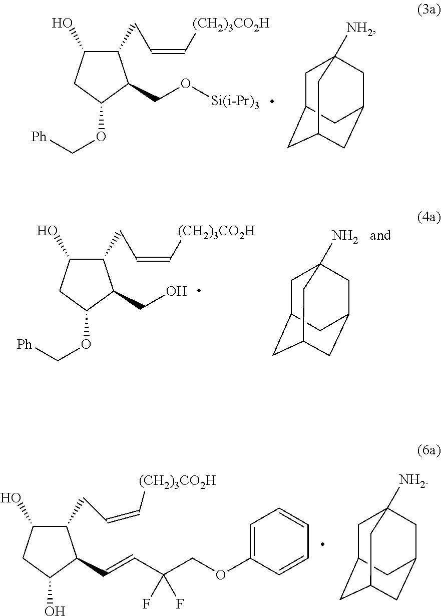

Illustrative embodiments of the present invention provide a crystalline salt described herein having the Formula 3a.

Illustrative embodiments of the present invention provide a crystalline salt described herein wherein the salt is characterized by a Powder X-Ray Diffraction (PXRD) diffractogram comprising a peak, expressed in degrees two-theta, at 11.0+/−0.2.

Illustrative embodiments of the present invention provide a crystalline salt described herein wherein PXRD diffractogram further comprises at least four peaks, expressed in degrees two-theta, selected from the group consisting of: 3.7+/−0.2, 7.5+/−0.2, 8.1+/−0.2, 9.5+/−0.2, 12.9+/−0.2, 13.6+/−0.2, 15.1+/−0.2, 15.7+/−0.2, 17.0+/−0.2 and 20.5+/−0.2.

Illustrative embodiments of the present invention provide a crystalline salt described herein wherein the salt is characterized by a Differential Scanning Calorimetry (DSC) thermogram comprising an endothermic peak having a peak onset at approximately 78° C.

Illustrative embodiments of the present invention provide a crystalline salt described herein having the Formula 4a.

Illustrative embodiments of the present invention provide a crystalline salt described herein wherein the salt is Form APO-I characterized by a Powder X-Ray Diffraction (PXRD) diffractogram comprising a peak, expressed in degrees two-theta, at approximately 5.6+/−0.2.

Illustrative embodiments of the present invention provide a crystalline salt described herein wherein the PXRD diffractogram further comprises at least four peaks, expressed in degrees two-theta, selected from the group consisting of: 7.5+/−0.2, 11.2+/−0.2, 15.2+/−0.2, 15.9+/−0.2, 16.9+/−0.2, 18.3+/−0.2, 18.9+/−0.2, 19.4+/−0.2, 21.2+/−0.2 and 23.7+/−0.2.

Illustrative embodiments of the present invention provide a crystalline salt described herein wherein the salt is characterized by a Differential Scanning Calorimetry (DSC) thermogram comprising an endothermic peak with a peak onset at approximately 117° C.

Illustrative embodiments of the present invention provide a crystalline salt described herein having a water content of less than about 0.5 wt %.

Illustrative embodiments of the present invention provide a crystalline salt described herein wherein the salt is Form APO-II characterized by a Powder X-Ray Diffraction (PXRD) diffractogram comprising a peak, expressed in degrees two-theta, at approximately 14.2+/−0.2.

Illustrative embodiments of the present invention provide a crystalline salt described herein wherein the PXRD diffractogram further comprises at least four peaks, expressed in degrees two-theta, selected from the group consisting of: 7.8+/−0.2, 10.0+/−0.2, 13.6+/−0.2, 15.5+/−0.2, 17.1+/−0.2, 17.9+/−0.2, 18.4+/−0.2, 19.5+/−0.2, 20.0+/−0.2, and 22.2+/−0.2.

Illustrative embodiments of the present invention provide a crystalline salt described herein wherein the molar ratio of water to the salt is 0.5 to 1.

Illustrative embodiments of the present invention provide a crystalline salt described herein having the Formula 6a.

Illustrative embodiments of the present invention provide a crystalline salt described herein wherein the salt is characterized by a Powder X-Ray Diffraction (PXRD) diffractogram comprising a peak, expressed in degrees two-theta, at approximately 6.0+/−0.2.

Illustrative embodiments of the present invention provide a crystalline salt described herein wherein the PXRD diffractogram further comprises at least four peaks, expressed in degrees two-theta, selected from the group consisting of 10.5+/−0.2, 12.1+/−0.2, 14.4+/−0.2, 16.0+/−0.2, 17.4+/−0.2, 18.3+/−0.2, 18.7+/−0.2, 19.4+/−0.2, 20.8+/−0.2, and 21.7+/−0.2.

Illustrative embodiments of the present invention provide a crystalline salt described herein wherein the molar ratio of water to the salt is 1.5 to 1.

Illustrative embodiments of the present invention provide a process for the preparation of a salt of formula 6a:

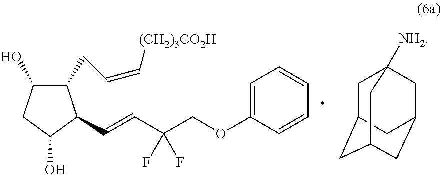

the process comprising: i) combining 1-adamantanamine, water, an organic solvent selected from the group consisting of ethers, esters, ketones and aromatic hydrocarbons and a compound of Formula 6:

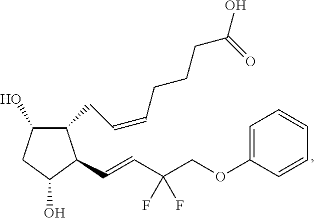 [SMILES: O=C(O)CCC/C=C\C[C@@H]1[C@@H](/C=C/C(F)(F)COc2ccccc2)[C@H](O)C[C@@H]1O ; ","]

thereby obtaining a suspension; and ii) isolating the solid salt of Formula 6a from the suspension.

Illustrative embodiments of the present invention provide a process described herein wherein the suspension is maintained for at least 30 minutes at a temperature of at least 40° C. prior to isolating the solid salt of Formula 6a from the suspension.

Illustrative embodiments of the present invention provide a process described herein wherein isolating comprises drying in vacuo at a temperature below 50° C.

Illustrative embodiments of the present invention provide a process for the preparation of a salt of Formula 3a:

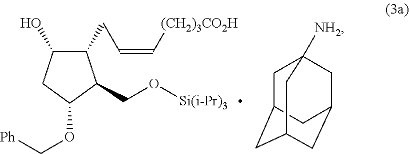

the process comprising: a) combining, in a hydrocarbon solvent selected from the group consisting of heptane, pentane and cyclohexane, 1-adamantanamine and a compound of Formula 3:

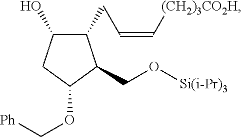

thereby obtaining a suspension; and b) isolating the solid salt of Formula 3a from the suspension.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate some embodiments of the invention,

FIG. 1 is a Powder X-Ray Diffraction (PXRD) diffractogram of form APO-I of the salt of Formula 3a as prepared in Example 3.

FIG. 2 is a Differential Scanning Calorimetry (DSC) thermogram of form APO-I of the salt of Formula 3a as prepared in Example 3.

FIG. 3 is a Powder X-Ray Diffraction (PXRD) diffractogram of form APO-I of the salt of Formula 4a as prepared in Example 4.

FIG. 4 is a Differential Scanning Calorimetry (DSC) thermogram of form APO-I of the salt of Formula 4a as prepared in Example 4.

FIG. 5 is a Powder X-Ray Diffraction (PXRD) diffractogram of form APO-II of the salt of Formula 4a as prepared in Example 6.

FIG. 6 is a Powder X-Ray Diffraction (PXRD) diffractogram of form APO-I of the salt of Formula 6a as prepared in Example 15.

### DETAILED DESCRIPTION

When used in reference to a diffractogram, a spectrum and/or data presented in a graph, the term “substantially similar” means that the subject diffractogram, spectrum and/or data presented in a graph encompasses all diffractograms, spectra and/or data presented in graphs that vary within acceptable boundaries of experimentation that are known to a person of skill in the art. Such boundaries of experimentation will vary depending on the type of the subject diffractogram, spectrum and/or data presented in a graph, but will nevertheless be known to a person of skill in the art.

When used in reference to a peak in a powder X-ray diffraction (PXRD) diffractogram, the term “approximately” means that the peak may vary by ±0.2 degrees 2-theta of the subject value.

When used in reference to a peak in a DSC thermogram, the term “approximately” means that the peak may vary by ±1° C. of the subject value.

As used herein, the term “about” means close to and that variation from the exact value that follows the term within amounts that a person of skill in the art would understand to be reasonable. In particular, when the term “about” is used with respect to temperature, a variation of +/−5° C. is often acceptable.

As used herein, the term “volumes” refers to the parts of solvent or liquids by volume (mL) with respect to the weight of solute (g). For example, when an experiment is conducted using 1 g of starting material and 100 mL of a solvent, it is said that 100 volumes of that solvent are used.

As used herein, when referring to a diffractogram, spectrum and/or to data presented in a graph, the term “peak” refers to a feature that one skilled in the art would recognize as not attributable to background noise.

Multi-component solid forms comprising more than one type of molecule, such as hydrates may have some variability in the exact molar ratio of their components depending on a variety of conditions understood to a person of skill in the art. For example, a molar ratio of components within a solvate provides a person of skill in the art information as to the general relative quantities of the components of the solvate and, in many cases, the molar ratio may vary by plus or minus 20% from a stated range. For example, a molar ratio of 1 to 1.5 is understood to include the ratio 1 to 1.2 as well as 1 to 1.8 as well as all of the individual ratios in between.

As used herein, the term “water content” refers to the amount of water present when measured by Karl Fischer (KF) analysis, expressed as a wt %.

As used herein, when referring to a solvent content, including water, the term “weight percentage” (wt %) refers to the ratio: weight part/weight whole, expressed as a percentage. For example, a 100 g sample of salt 4a containing 1.5 g water is said to contain 1.5 wt % water.

Depending on the nature of the methodology applied and the scale selected to display results obtained from an X-ray diffraction analysis, an intensity of a peak obtained may vary quite dramatically. For example, it is possible to obtain a relative peak intensity of 1% when analyzing one sample of a substance, but another sample of the same substance may show a much different relative intensity for a peak at the same position. This may be due, in part, to the preferred orientation of the sample and its deviation from the ideal random sample orientation, sample preparation and the methodology applied. Such variations are known and understood by a person of skill in the art.

Lubiprostone may be prepared according to the route shown in Scheme 1. In this route, a compound of Formula 3 is deprotected to give a compound of Formula 4, which may be converted to Lubiprostone. WO 2010/083597 A1, for instance, discloses conditions suitable for preparing Lubiprostone according to the route shown in Scheme 1.

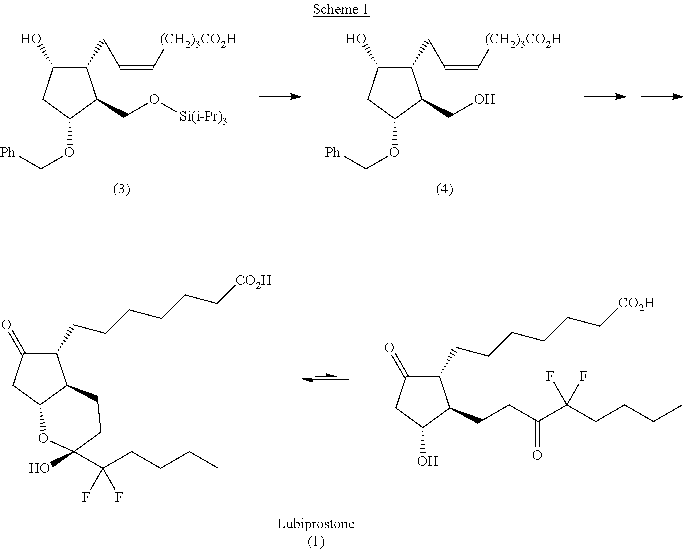

A compound of Formula 6 is an intermediate useful in the preparation of Tafluprost (as disclosed in, for example, Matsumura, Y. et al. in Tet. Lett. 2004, 45, 1527) as shown in Scheme 2.

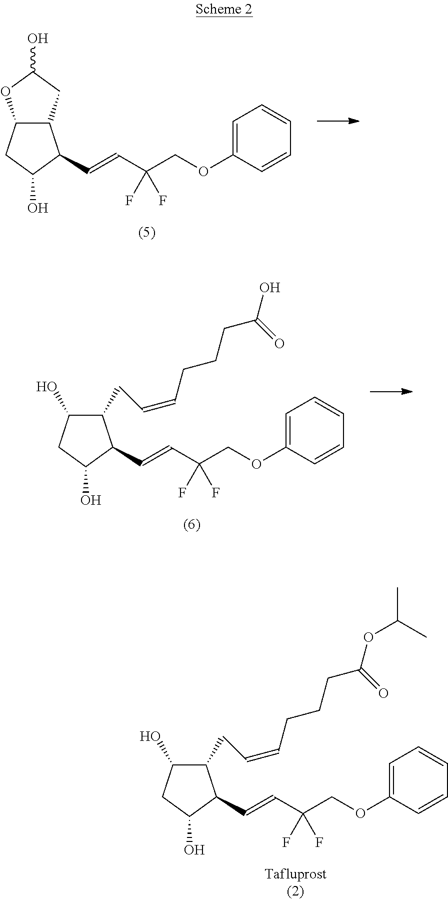

Compounds 3, 4 and 6 are oils under ambient conditions, like many of the intermediates in the reported syntheses of Lubiprostone and Tafluprost. Oils are generally more difficult to handle with respect to purification and/or handling than solids, especially on an industrial scale. Since the preparation of each of Lubiprostone and Tafluprost involves a relatively large number of steps, difficulties in the purification and/or the handling of intermediates, such as intermediate oils, can create obstacles to achieving the high purity required for pharmaceutical products.

An embodiment of the present invention provides a crystalline salt of a prostaglandin analog intermediate having a formula selected from the group consisting of:

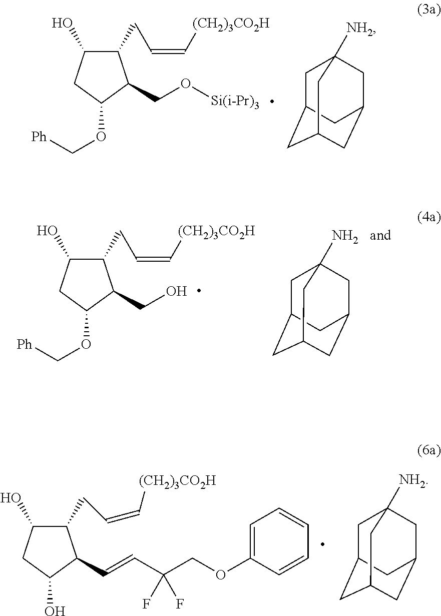

Some embodiments of the present invention relate to a solid form of the salt of Formula 3a, termed herein Form APO-I of the salt of Formula 3a.

In some embodiments of the present invention, Form APO-I of the salt of Formula 3a may be characterized by a PXRD diffractogram comprising a peak at 11.0+/−0.2 degrees two-theta.

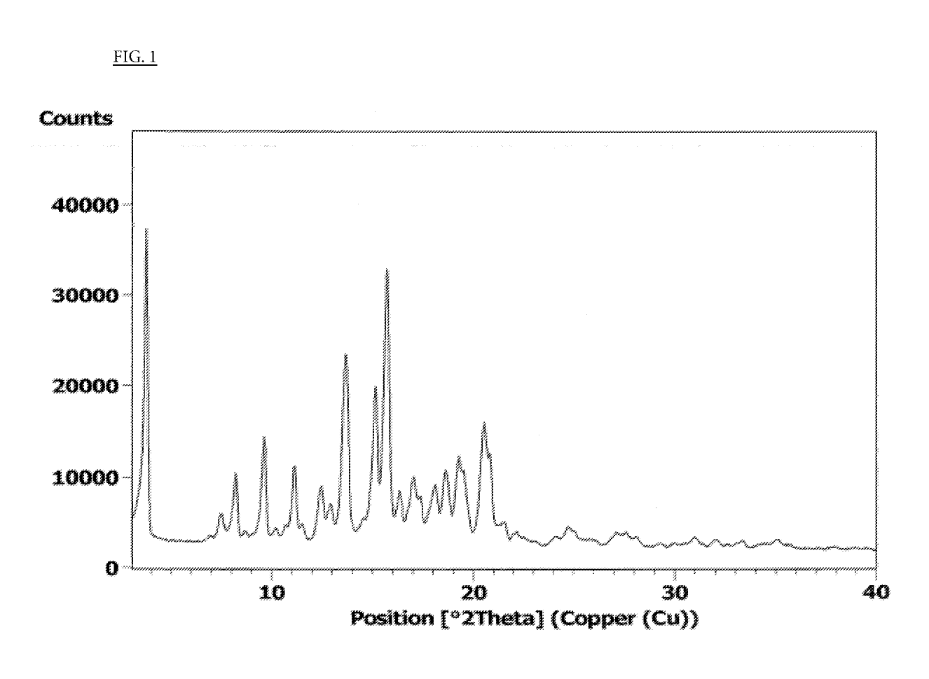

An illustrative PXRD diffractogram of Form APO-I of the salt of Formula 3a is shown in FIG. 1.

In some embodiments, Form APO-I of the salt of Formula 3a may have a peak at any one or more of the values expressed in degrees 2-theta given in Table 1. Although values are given in the tables below, APO-I of the salt of Formula 3a may be defined by the claimed peaks and a particular claim may be limited to one peak only, or several peaks. The form APO-1 of the salt of Formula 3a does not have to include all or even many of the peaks listed in Table 1. Some illustrative and non-limiting possible observations regarding relative intensities of the peaks are set out in Table 1.

TABLE 1
PXRD peaks and relative peak intensities
of Form APO-I of the salt of Formula 3a

| Angle 2-theta | Relative intensity % |
|---|---|
| 3.72 | 100.00 |
| 7.47 | 8.74 |
| 8.06 | 10.12 |
| 9.48 | 16.6 |
| 11.04 | 16.42 |
| 12.32 | 7.41 |
| 12.45 | 14.24 |
| 12.89 | 12.54 |
| 13.59 | 46.82 |
| 14.51 | 3.83 |
| 15.09 | 43.31 |
| 15.68 | 79.85 |
| 16.32 | 14.88 |
| 17.01 | 21.98 |
| 17.37 | 8.38 |
| 18.05 | 19.14 |
| 18.57 | 18.61 |
| 18.73 | 13.48 |
| 19.23 | 24.82 |
| 19.54 | 18.19 |
| 20.50 | 39.59 |
| 20.84 | 23.03 |

In some embodiments, Form APO-I of the salt of Formula 3a may be characterized by a DSC thermogram comprising an endothermic peak with a peak onset at approximately 78° C.

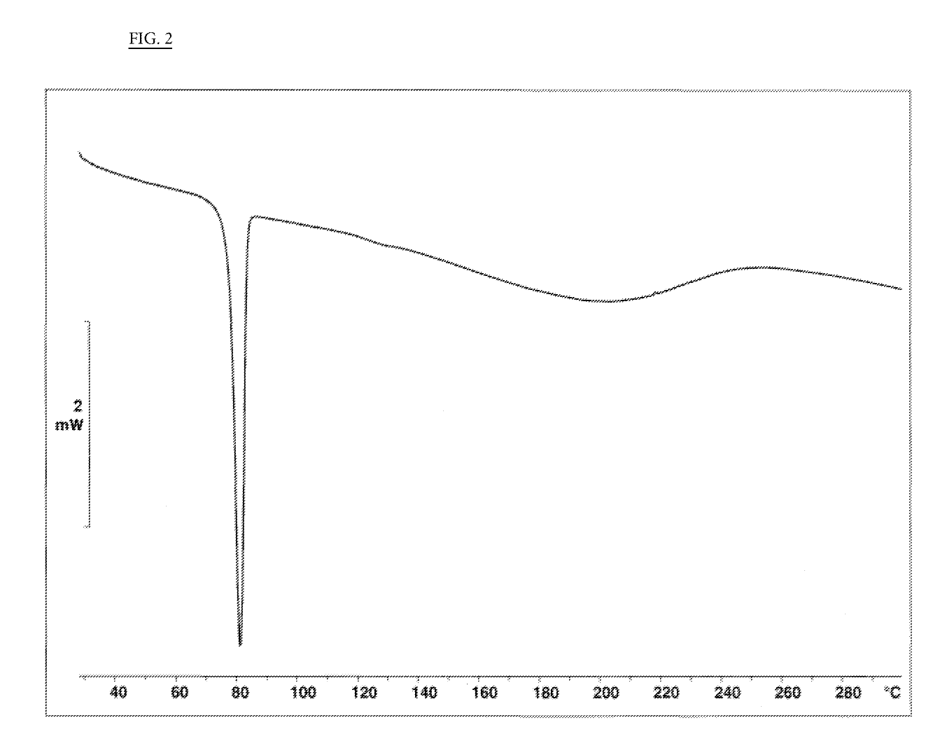

An illustrative DSC thermogram of Form APO-I of the salt of Formula 3a is shown in FIG. 2.

Another aspect of the present invention provides a process for the preparation of the salt of Formula 3a comprising:

a) combining a compound of Formula 3:

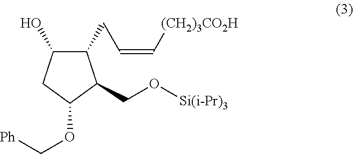

and 1-adamantanamine in a hydrocarbon solvent and obtaining a suspension;

b) isolating the solid from the suspension to yield the salt of Formula 3a.

The hydrocarbon solvent may be selected from the group consisting of heptane, pentane and cyclohexane.

The amount of solvent that may be used is often from about 1 volume to about 10 volumes with respect to the weight of the compound of Formula 3.

In some embodiments, the present invention provides a crystalline form of the salt of Formula 4a.

In some embodiments, the present invention provides a crystalline form of the salt of Formula 4a, having a water content of less than about 0.5 wt %, termed herein Form APO-I of the salt of Formula 4a.

In some embodiments of the present invention, Form APO-I of the salt of Formula 4a may be characterized by a PXRD diffractogram comprising a peak at 5.6+/−0.2 degrees two-theta.

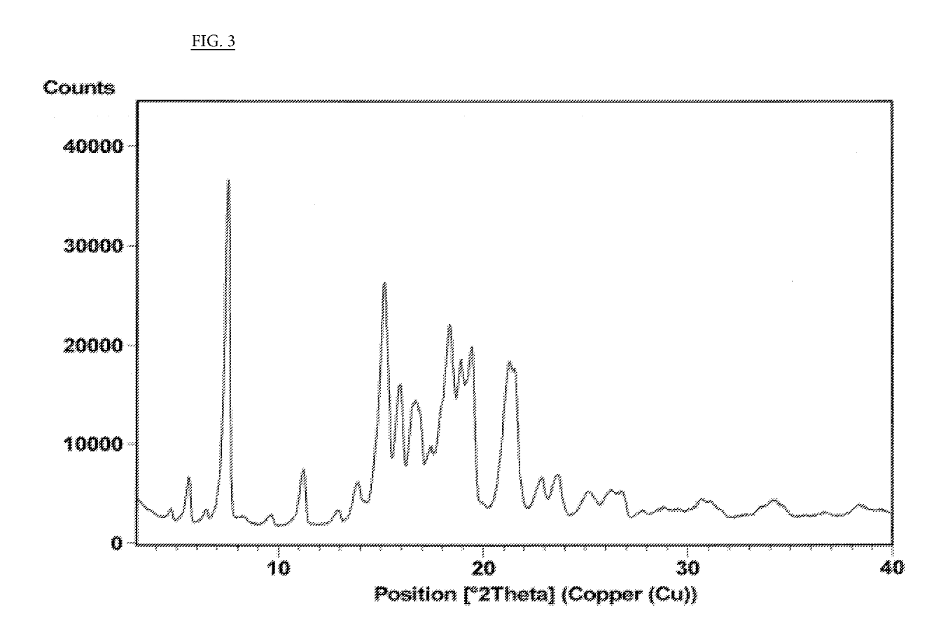

An illustrative PXRD diffractogram of Form APO-I of the salt of Formula 4a is shown in FIG. 3.

In some embodiments, Form APO-I of the salt of Formula 4a may have a peak at any one or more of the values expressed in degrees 2-theta given in Table 2. Although values are given in the tables below, APO-I of the salt of Formula 4a may be defined by the claimed peaks and a particular claim may be limited to one peak only, or several peaks. The form APO-I of the salt of Formula 4a does not have to include all or even many of the peaks listed in Table 2. Some illustrative and non-limiting possible observations regarding relative intensities of the peaks are set out in Table 2.

TABLE 2
PXRD peaks and relative peak intensities
of Form APO-I of the salt of Formula 4a

| Angle 2-theta | Relative intensity % |
|---|---|
| 4.69 | 2.66 |
| 5.56 | 13.42 |
| 6.42 | 3.76 |
| 7.47 | 100.00 |
| 9.56 | 3.05 |
| 11.16 | 17.13 |
| 12.87 | 4.15 |
| 13.80 | 12.12 |
| 13.92 | 11.12 |
| 15.19 | 72.5 |
| 15.92 | 43.49 |
| 16.91 | 32.14 |
| 17.87 | 32.66 |
| 18.34 | 60.94 |
| 18.93 | 44.87 |
| 19.41 | 56.53 |
| 21.20 | 46.8 |
| 22.75 | 12.57 |
| 23.68 | 12.23 |
| 25.09 | 8.25 |

In some embodiments, Form APO-I of the salt of Formula 4a may be characterized by a DSC thermogram comprising an endothermic peak with a peak onset at approximately 117° C.

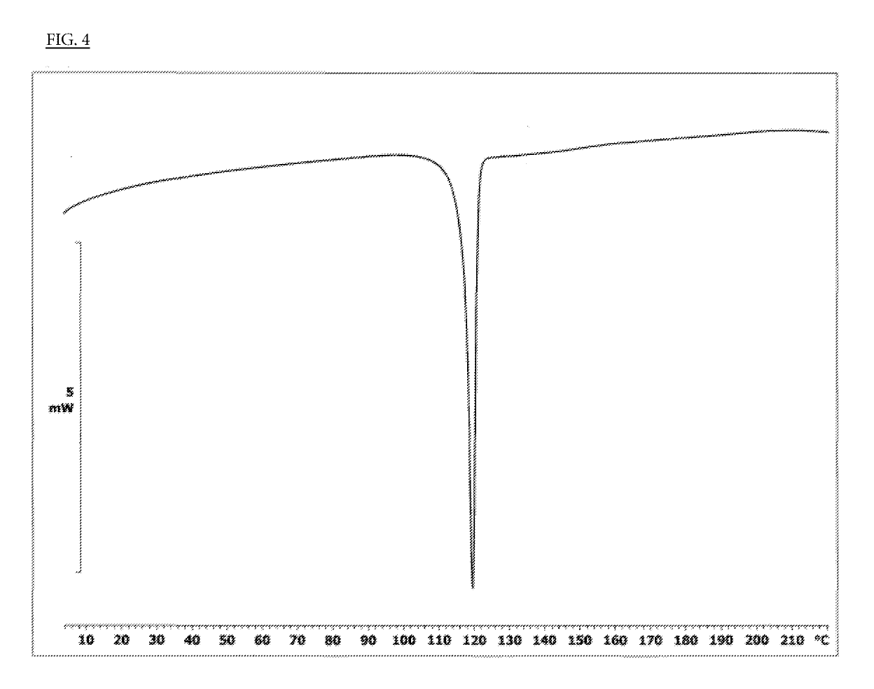

An illustrative DSC thermogram of Form APO-I of the salt of Formula 4a is shown in FIG. 4.

In some embodiments, the present invention provides a hemi-hydrate form of the salt of Formula 4a, termed herein Form APO-II of the salt of Formula 4a, wherein the molar ratio of water to the salt of Formula 4a is 0.5 to 1.

In some embodiments of the present invention, Form APO-II of the salt of Formula 4a may be characterized by a PXRD diffractogram comprising a peak at 14.2+/−0.2 degrees two-theta.

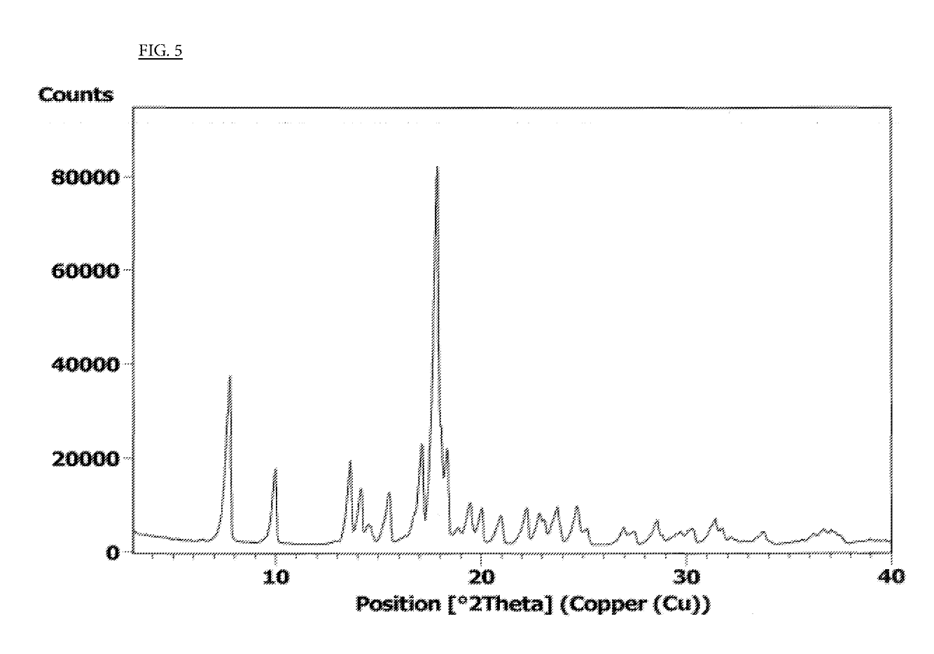

An illustrative PXRD diffractogram of Form APO-II of the salt of Formula 4a is shown in FIG. 5.

In some embodiments, Form APO-II of the salt of Formula 4a may have a peak at any one or more of the values expressed in degrees 2-theta given in Table 3. Although values are given in the tables below, APO-II of the salt of Formula 4a may be defined by the claimed peaks and a particular claim may be limited to one peak only, or several peaks. The form APO-II of the salt of Formula 4a does not have to include all or even many of the peaks listed in Table 3. Some illustrative and non-limiting possible observations regarding relative intensities of the peaks are set out in Table 3.

TABLE 3
PXRD peaks and relative peak intensities
of form APO-II of the salt of Formula 4a

| Angle 2-theta | Relative intensity % |
|---|---|
| 7.76 | 56.35 |
| 9.96 | 28.12 |
| 13.64 | 25.97 |
| 14.17 | 13.96 |
| 14.64 | 5.83 |
| 15.52 | 18.44 |
| 17.14 | 28.02 |
| 17.87 | 100.00 |
| 18.37 | 25.34 |
| 18.85 | 4.99 |
| 19.48 | 5.64 |
| 20.04 | 10.92 |
| 20.96 | 10.27 |
| 22.21 | 12.57 |
| 22.83 | 12.4 |
| 23.08 | 6.43 |
| 23.88 | 3.47 |
| 24.63 | 12.53 |
| 25.14 | 6.68 |

In some embodiments, the present invention provides a hydrate form of the salt of Formula 6a, termed herein Form APO-I of the salt of Formula 6a, wherein the molar ratio of water to the salt of Formula 6a is 1.5 to 1.

In some embodiments of the present invention, Form APO-I of the salt of Formula 6a may be characterized by a PXRD diffractogram comprising a peak at 6.0+/−0.2 degrees two-theta.

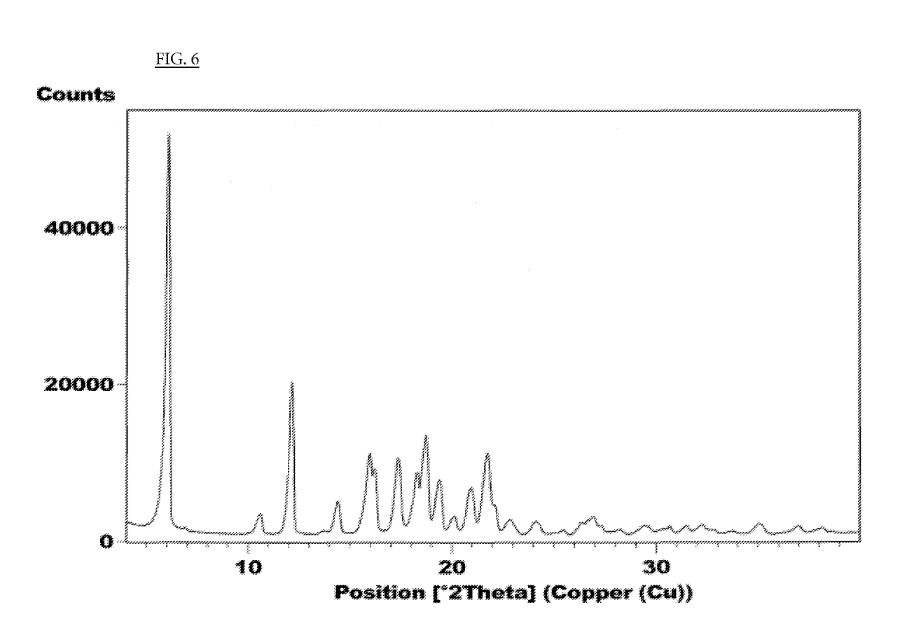

An illustrative PXRD diffractogram of Form APO-I of the salt of Formula 6a is shown in FIG. 6.

In some embodiments, Form APO-I of the salt of Formula 6a may have a peak at any one or more of the values expressed in degrees 2-theta given in Table 4. Although values are given in the tables below, APO-I of the salt of Formula 6a may be defined by the claimed peaks and a particular claim may be limited to one peak only, or several peaks. The form APO-I of the salt of Formula 6a does not have to include all or even many of the peaks listed in Table 4. Some illustrative and non-limiting possible observations regarding relative intensities of the peaks are set out in Table 4.

TABLE 4
PXRD peaks and relative peak intensities
of Form APO-I of the salt of Formula 6a

| Angle 2-theta | Relative intensity % |
|---|---|
| 6.01 | 100.00 |
| 10.54 | 5.56 |
| 12.12 | 32.89 |
| 14.36 | 8.96 |
| 15.97 | 16.12 |
| 16.21 | 13.72 |
| 17.37 | 15.84 |
| 18.26 | 15.3 |
| 18.69 | 23.88 |
| 19.43 | 10.33 |
| 20.06 | 4.61 |
| 20.84 | 9.53 |
| 21.69 | 21.1 |
| 22.11 | 5.37 |
| 22.83 | 3.49 |
| 24.07 | 2.99 |

Another aspect of the present invention is a process for the preparation of the salt of Formula 6a comprising:
i) combining 1-adamantanamine, water, a compound of Formula 6

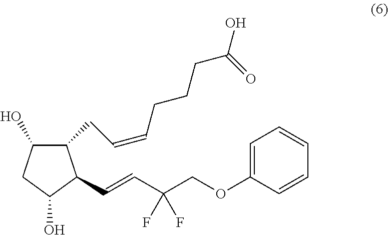 [SMILES: O=C(O)CCC/C=C\C[C@@H]1[C@@H](/C=C/C(F)(F)COc2ccccc2)[C@H](O)C[C@@H]1O]

and an organic solvent selected from the group consisting of ethers, esters, ketones and aromatic hydrocarbons to yield a mixture;
ii) maintaining the mixture, if necessary, to allow for the formation of a suspension of a salt of Formula 6a;
iii) isolating the salt from the suspension to yield the salt of Formula 6a.

The organic solvent may be selected from the group consisting of ethers such as tetrahydrofuran and methyl t-butyl ether, esters such as ethyl acetate and isopropyl acetate, ketones such as acetone and methyl ethyl ketone and aromatic hydrocarbons such as toluene. The amount of organic solvent often varies from about 1 volume to about 10 volumes with respect to the weight of a compound of Formula 6, with volumes in the lower range more suitable for the ketones. The amount of water often varies from about 4 volumes to about 10 volumes with respect to the weight of the compound of Formula 6. The water is particularly effective for dissolving a major by-product of the reaction to generate the compound of Formula 6, 5-(diphenylphosphoryl)pentanoic acid, while the salt 6a shows lower solubility in these conditions and so can be isolated and purified from this impurity by filtration.

A mixture obtained in step i) may initially consist of a light suspension consisting primarily of 1-adamantanamine. The mixture obtained in step i) may be maintained for a time to allow for more complete reaction of a compound of Formula 6 with 1-adamantanamine to generate a suspension comprising the salt of Formula 6a. Often the mixture is maintained for a period of at least about 30 minutes prior to isolation. The mixture may also be heated, if desired, to a temperature between about 40° C. and the boiling point of the solvent to aid salt formation.

The 1-adamantanamine may be provided as the free amine or as an acid salt, such as the HCl salt, which is liberated by treatment with a base, such as sodium hydroxide. In the latter case, the base may be provided as a solution in water, which may contribute the water for the process.

The salt of Formula 6a may be dried in vacuo at a temperature below about 50° C. The salt will melt above 65° C.

### EXAMPLES

The following examples are illustrative of some of the embodiments of the invention described herein. These examples do not limit the spirit or scope of the invention in any way.

### Powder X-Ray Diffraction Analysis:

Data were acquired on a PANanalytical X-Pert Pro MPD diffractometer with fixed divergence slits and an X'Celerator RTMS detector. The diffractometer was configured in Bragg-Brentano geometry; data was collected over a 2-theta range of 3 to 40 degrees using CuK-alpha radiation at a power of 40 mA and 45 kV. CuK-beta radiation was removed using a divergent beam nickel filter. A step size of 0.017 degrees was used. Samples were rotated to reduce preferred orientation effects. Samples were lightly ground prior to analysis.

### Differential Scanning Calorimetry Analysis:

The DSC thermograms were collected on a Mettler-Toledo 821e instrument. Samples (1-2 mg) were weighed into a 40 μL aluminum pan and were crimped closed with an aluminum lid having a 50 μm hole. The samples were analyzed under a flow of nitrogen (ca. 50 mL/min) at a scan rate of 10° C./minute.

### Example 1: Preparation of the Salt of Formula 3a

To a solution of (4-carboxybutyl)triphenylphosphonium bromide (100.23 g, 225 mmol) in tetrahydrofuran (380 mL) was charged potassium t-butoxide (50.68 g, 451 mmol) at about 0° C. The reaction mixture was allowed to warm to about 25° C. To the resulting brown color solution was dropwise added a solution of (2S*/R*,3aR,4S,5R,6aS)-5-benzyloxy-4-Rtriisopropylsilyloxy)methypexahydro-2H-cyclopenta[b]furan-2-ol (38 g, 90 mmol) in tetrahydrofuran (76 mL) at about 25° C. The resulting orange color solution was stirred at about 25° C. for about 3 hours, followed by a check by thin-layer chromatography for reaction completion (disappearance of starting material). After reaction completion was confirmed, the mixture was quenched with water (38 mL) at about 30° C. (exotherm) and filtered. The solid was washed with ethyl acetate (2×38 mL). The filtrate was concentrated in vacuo to dryness, followed by addition of ethyl acetate (228 mL) and 5% aqueous HCl (190 mL). The layers were separated and the organic phase was washed with water (2 vol) and concentrated in vacuo. The crude free acid was then purified by column chromatography (heptane/ethyl acetate eluent, 80/20). The combined fractions containing the free acid were evaporated to afford an oil. A sample of the oil (1.0 g) was dissolved in heptane (10 mL) followed by the addition of 1-adamantanamine (0.30 g, 1.98 mmol). The suspension obtained was stirred for about 1.5 hours at about 25° C., filtered and washed with heptane and dried in vacuo to give the the salt 3a as a white to off-white crystalline solid. ¹H-NMR, DMSO-d₆, 400 mHz, delta 7.36-7.20 m (5H), 6.78 bs (3H), 5.47-5.37 m (1H), 5.37-5.27 m (1H), 4.47 d (J=11.9 Hz) and 4.37 d (J=12.0 Hz) (2H), 4.02-3.96 m (1H), 3.88-3.80 m (1H), 3.80-3.63 m (2H), 2.26-2.05 m (2H), 2.05-1.94 m (8H), 1.94-1.87 m (1H), 1.76-1.65 m (7H), 1.65-1.46 m (9H), 1.10-0.90 m (21H). ¹³C-NMR, DMSO-d₆, 100 MHz, delta 176.3 138.9, 129.6, 128.8, 128.0, 127.2, 127.1, 79.9, 70.7, 70.3, 62.3, 51.1, 49.0, 44.7, 41.6, 39.9, 36.6, 35.5, 28.6, 26.8, 26.0, 25.4, 17.8, 11.4.

### Example 2: Preparation of the Salt of Formula 3a

To a solution of (4-carboxybutyl)triphenylphosphonium bromide (167.65 g, 377 mmol) in tetrahydrofuran (572 mL) was charged potassium t-butoxide (84.76 g, 755 mmol) at 0-5° C. The reaction mixture was allowed to warm to about 25° C. To the resulting brown color solution was dropwise added a solution of (2S*/R*,3aR,4S,5R,6aS)-5-benzyloxy-4-[(triisopropylsilyloxy)methyl]hexahydro-2H-cyclopenta[b]furan-2-ol (63.56 g, 151 mmol) in THF (127 mL) at about 25° C. The resulting orange color solution was stirred at about 25° C. for about 1.5 hours, followed by a check by thin-layer chromatography for reaction completion (disappearance of starting material). After reaction completion was confirmed, the mixture was filtered and the solid was washed with heptane (63.5 mL). The filtered damp caked was slurried with a 1/2 (by volume) mixture of ethyl acetate to heptane (378 mL), filtered and the solid was washed with a 1/2 (by volume) mixture ethyl acetate to heptane mixture (127 mL). The pH of the filtrate was adjusted to about 2.5 using aqueous HCl (2 M). The layers were separated and the aqueous layer was extracted with ethyl acetate (126 mL). The combined organic phases were washed with water (2×127 mL) and the organic layer was concentrated in vacuo to dryness. The crude free acid was then purified by column chromatography (heptane/ethyl acetate eluent, 80/20). The oil obtained was dissolved in heptane (265 mL) followed by the addition of 1-adamantanamine (19.91 g). The suspension was stirred for about 17 hours at about 25° C., before it was cooled to about 0° C. The suspension was stirred for about 2.5 hours at about 0° C., filtered, washed with heptane (33 mL) and dried in vacuo at about 25° C. to afford salt 3a as a white to off-white crystalline solid (76.7 g).

### Example 3: Preparation of Form APO-I of the Salt of Formula 3a

To a solution of compound 3 (83.0 g oil, 75.0 g corrected by ¹H NMR), prepared according to the procedure of Example 2, in heptane (300 mL) was charged 1-adamantanamine (22.47 g). The solution was allowed to stir at about 25° C. for 2.5 hours. The resulting suspension was cooled to about 0° C. and maintained for 2 hours. The solid was isolated by filtration, washed with heptane (75 mL) and dried in vacuo to afford salt 3a (91.27 g, 93.4% yield). A PXRD diffractogram and DSC thermogram of a sample prepared by this method are shown in FIG. 1 and FIG. 2, respectively.

### Example 4: Preparation of Form APO-I of the Salt of Formula 4a

To a solution of the compound of Formula 3 (46.0 g, 91 mmol) in tetrahydrofuran (138 mL) was charged tetrabutyl ammonium fluoride (1.0 M in tetrahydrofuran, 364.5 mL). The resulting solution was stirred at about 25° C. for about 12 hours, followed by a check for reaction completion by thin-layer chromatography (disappearance of starting material). Upon reaction completion, the reaction mixture was evaporated in vacuo to a minimum volume, followed by charging deionized water (184 mL) and heptane (184 mL). The pH was then adjusted to about 11.5 using aqueous NaOH. After stirring at about 25° C. for about 20 minutes, the two phases were separated; the organic phase contained the silyl by-product and was discarded as waste. The pH of the aqueous phase was adjusted to about 2.5 using aqueous HCl (1 M) and extracted with ethyl acetate (2×150 mL). The combined extracts were washed with water (100 mL) and concentrated in vacuo to yield an oil (38 g). The oil was re-partitioned in heptane and water at pH 11.0. The layers were separated, the pH of the aqueous phase was adjusted to pH 3 and the aqueous phase was extracted with ethyl acetate. The combined ethyl acetate extracts were washed with water and evaporated to yield compound 4 as an oil (30.4 g). Ethyl acetate (150 mL) was charged to the oil to form a solution. To the solution was charged 1-adamantanamine (12.8 g, 1.0 mole equivalents based on the available amount of the free acid) and the mixture was stirred for about 30 minutes to observe precipitation formation. Heptane (230 mL) was charged and the resulting slurry was agitated at about 60° C. for about 2 hours. A 1/1 (by volume) mixture of ethyl acetate to heptane was charged. The slurry was further maintained at about 60° C. for 1.5 hours and then cooled to about 25° C. for about 0.5 hour. The solid was isolated by filtration and dried in vacuo (50-10 Torr) at about 50° C. for 15.5 hours to afford the 1-adamantanamine salt of (5Z)-7-[(1R,2S,3R,5S)-5-hydroxy-2-(hydroxymethyl)-3-(phenylmethoxy)cyclopentyl]-5-heptenoic acid (4a) as a white to off-white crystalline solid (37.6 g). The sample was stored in a tight, light-resistant container under nitrogen prior to analysis. Elemental analysis: Cal C 72.11, H 9.08, N2.80; found C71.96, H 8.81, N 2.92. ¹H-NMR, DMSO-d₆, 400 mHz, delta 7.40-7.20 m (5H), 5.65 bs (2H), 5.50-5.38 m (1H), 5.38-5.28 m (1H), 4.50-4.38 m (2H), 4.00-3.92 m (1H), 3.85-3.75 m (1H), 3.51 dd (J=10.8, 4.3 Hz) and 3.39 dd (J=10.8, 5.1 Hz) (2H), 2.22-2.08 m (2H), 2.08-1.90 m (8H), 1.90-1.78 m (1H), 1.72-1.60 m (7H), 1.60-1.40 m (9H). ¹³C-NMR, DMSO-d₆, 100 MHz, delta 176.0, 139.2, 129.5, 129.3, 128.1, 127.3, 127.1, 80.6, 70.9, 70.1, 60.8, 51.2, 48.8, 45.2, 42.3, 40.1, 36.0, 35.5, 28.7, 26.7, 25.7, 25.5. A PXRD diffractogram and DSC thermogram of a sample prepared by this method is shown in FIG. 3 and FIG. 4, respectively.

### Example 5: Preparation of Form APO-I of the Salt of Formula 4a

To the compound of Formula 4 (710 g), prepared as in Example 4, was charged ethyl acetate (7000 mL) and water (1500 mL). The mixture was cooled to about 0° C. and the pH was adjusted to about 3 using aqueous HCl (5N) while controlling the internal temperature between 0-10° C. The organic phase was separated and the water phase was extracted with ethyl acetate (5000 mL). The combined organic phase was concentrated in vacuo to about 2000 mL followed by an additional charge of ethyl acetate (3000 mL). The mixture was further concentrated to about 1000 mL. To the residue (about 900 g) was charged ethyl acetate (7000 mL) and 1-adamantanamine (236.8 g). The reaction mixture was heated to gentle reflux (75-80° C.) and stirred for 1 hour. The reaction mixture was then cooled to about 25° C. and stirred at this temperature for 17 hours. The mixture was filtered, washed with ethyl acetate (2×2000 mL) and dried in vacuo at about 45° C. for about 12 hours to yield the salt of Formula 4a (651 g, 91% yield). This lot was packaged after drying in an inner antistatic polyethylene bag sealed with a cable tie encased within a heat sealed composite polyethylene/aluminum foil outer bag under nitrogen.

### Example 6: Preparation of Form APO-II of the Salt of Formula 4a

A sample (0.34 g) of Form APO-I salt 4a obtained in Example 5 was placed in an open vial (25 mL) in a desiccator containing distilled water (20 mL) in an open beaker (50 mL) to expose the salt to humidity. The closed desiccator was maintained at about 20° C. for 10 days and the sample was analysed by KF, DSC and PXRD. KF (initial)=0.34 wt %, KF (final)=1.82 wt %. PXRD analysis demonstrated that the Form APO-I had converted to Form APO-II following exposure to these conditions. A PXRD diffractogram of a sample of Form APO-II is shown in FIG. 5.

### Example 7: Preparation of the Salt of Formula 6a

To a suspension of (4-carboxybutyl)triphenylphosphonium bromide (18.0 g, 40.6 mmol) in tetrahydrofuran (53 mL) was charged a solution of sodium bis(trimethylsilyl)amide in tetrahydrofuran (36 wt %, 41.4 g, 81.2 mmol) at about 5° C. The mixture was then warmed to about 20° C. After stirring for about 45 minutes, the mixture was cooled to about −5° C., and a solution of a compound of Formula 5 (5.3 g, 16.2 mmol) in toluene (about 5 mL) was charged dropwise while controlling the internal temperature to between −10-0° C. Upon reaction completion (by thin-layer chromatography), 5% brine (53 mL) was charged to quench the reaction. The aqueous phase was separated and extracted with methyl t-butyl ether (53 mL). The aqueous phase was cooled to about 5° C. and acidified to pH 1-2 with 36 wt % aqueous HCl before addition of methyl t-butyl ether (53 mL). After stirring for 30 minutes, the suspension was filtered and rinsed with pre-cooled (about 5° C.) methyl t-butyl ether (53 mL). The filtrate was separated and the organic phase was washed with 10% brine (2×30 mL). To the organic phase was charged 1-adamantanamine (3.4 g, 22.7 mmol), followed by water (26 mL), in one portion. The suspension was heated to about 55° C. and maintained for 30 minutes. After cooling to about 25° C., the suspension was stirred overnight. The solid was collected by filtration and the filter cake was washed with water (26 mL) and methyl t-butyl ether (53 mL). Crude salt 6a was obtained as a yellow to off-white solid (7.45 g, 77.9% yield, HPLC purity 97.85%) after drying in vacuo (38 Torr) at about 45° C. for 8 hours.

### Example 8: Purification of Crude Salt of Formula 6a

To the crude salt 6a (7.45 g), prepared as in Example 7, was charged water (60 mL) and acetone (8 mL). The mixture was heated to about 60° C. and maintained for 30 minutes before being cooled to about 25° C. After stirring for 1.5 hours, the suspension was filtered and the filter cake was rinsed with a mixed solvent (water/acetone: 8/1 (by volume) ratio, 15 mL) and dried in vacuo (38 Torr) at about 45° C. for 8 hours to give the purified product (6.86 g, 92.1% yield, HPLC purity 99.61%).

### Example 9: Preparation of the Salt of Formula 6a

To the salt 6a (6.14 g), prepared as in Example 8, was charged methyl t-butyl ether (61 mL) and acetone (6.1 mL). The mixture was heated to reflux (about 56° C.) and maintained for 30 minutes before being cooled to about 25° C. After stirring for 3 hours, the suspension was filtered and the filter cake was rinsed with a mixed solvent (methyl t-butyl ether/acetone: 10/1 (by volume) ratio, 18 mL) and dried in vacuo (38 Torr) at about 45° C. for 4 hours to give the purified product (5.80 g, 94.5% yield, HPLC purity 99.48%).

### Example 10: Preparation of the Salt of Formula 6a

To methanol (100 mL) was charged salt 6a (24.5 g). The mixture was stirred for 10 minutes and filtered. The filtrate was re-filtered through diatomite (1 g). The resulting filtrate was concentrated to dryness in vacuo (38 Torr) at 40° C. To the residue was charged methyl t-butyl ether (220 mL) and water (0.8 mL). The mixture was heated to reflux (about 56° C.) and then cooled to about 25° C. After stirring for 2 hours, the suspension was filtered and the filter cake was rinsed with methyl t-butyl ether (50 mL). The damp filter cake was dried in vacuo (38 Torr) at about 45° C. to give the dry product which was then grinded for 2 minutes to afford salt 6a as a white solid.

### Example 11: Preparation of the Salt of Formula 6a

To a suspension of (4-carboxybutyl)triphenylphosphonium bromide (67.9 g, 153.2 mmol) in tetrahydrofuran (200 mL) was charged a solution of sodium bis(trimethylsilyl)amide in tetrahydrafuran (37 wt %, 184 g, 371.4 mmol) at about 5° C. The mixture was then warmed to about 20° C. After 1 hour of stirring, the mixture was cooled to about −5° C., and a solution of a compound of Formula 5 (1.0 eq., 20.0 g, 61.3 mmol) in THF (60 mL) was charged dropwise while controlling the internal temperature to below about −5° C. Upon reaction completion by thin-layer chromatography, water (200 mL) was charged to quench the reaction. The aqueous phase was separated and extracted with isopropyl acetate (160 mL). The aqueous phase was cooled to about 5° C., before addition of isopropyl acetate (200 mL) and 36 wt % aqueous HCl (31.4 g) while maintaining the temperature below 10° C. After stirring for about 1 hour, the mixture was filtered and the filter cake was rinsed with pre-cooled (about 5° C.) isopropyl acetate (100 mL). The filtrate was separated and the organic phase was washed with 10% brine (3×100 mL). The organic phase had a mass of 309.8 g. To a portion (77.3 g solution, 5.0 g compound 5) of the organic phase was charged 1-adamantanamine hydrochloride (4.36 g, 23.2 mmol) and water (25 mL) followed by dropwise addition of aqueous NaOH (8.45%, 10.6 g, 22.4 mmol). The mixture was heated to 50-60° C. and maintained for 30 minutes. After cooling to about 25° C., the suspension was stirred overnight. The suspension was then cooled to about 5° C. and maintained for 2 hours. The solid was collected by filtration and the filter cake was washed with water (10 mL) and isopropyl acetate (20 mL). Crude salt 6a was obtained as a light yellow solid (7.50 g, 83.2% yield) after drying for 8 hours in vacuo (38 Torr).

### Example 12: Preparation of the Salt of Formula 6a

A portion (77.3 g solution, 5.0 g compound 5) of the 309.8 g of organic phase prepared in Example 11 was concentrated in vacuo (38 Torr) while maintaining the temperature below 45° C. to yield a yellow oil (11 g, ˜10 mL). To the oil was charged 1-adamantanamine hydrochloride (4.36 g, 23.2 mmol) and water (50 mL). An aqueous solution of NaOH (8.45%, 10.6 g, 22.4 mmol) was added dropwise. The mixture was heated to about 55° C. and maintained for 30 minutes. After cooling to about 25° C., the suspension was stirred overnight. The solid was collected by filtration and the filter cake was washed with water (10 mL) and isopropyl acetate (20 mL). Crude salt 6a was obtained after drying for 8 hours in vacuo (38 Torr) as a light yellow solid (7.46 g, 82.7% yield).

### Example 13: Preparation of Form APO-1 of the Salt of Formula 6a

To a slurry of (4-carboxybutyl)triphenylphosphonium bromide (13.25 g, 29.9 mmol) and tetrahydrofuran (39 mL) at about 5° C. was dropwise charged a solution of sodium bis(trimethylsilyl)amide in tetrahydrofuran (36 wt %, 35.7 g, 71.7 mmol) while maintaining the internal temperature below about 10° C. Upon addition completion, the resulting red slurry was warmed to about 20° C. After stirring for 1 hour, the slurry was cooled to about −5° C. A solution of a compound of Formula 5 in toluene (11.6 g, containing 3.9 g compound 5, 11.9 mmol) was added dropwise while maintaining the internal temperature below about 0° C. After the addition was completed, the solution was stirred at about −5° C. until thin-layer chromatography indicated reaction completion. Brine (5%, 40 mL) was added and the organic phase was separated. The aqueous phase was extracted with methyl t-butyl ether (40 mL), and then the aqueous phase was cooled to about 5° C. followed by charging methyl t-butyl ether (40 mL) and 36 wt % aqueous HCl (6 mL). The mixture was stirred for about 10 minutes. A precipitate was not observed so diatomite (0.2 g) was charged to accelerate the precipitation of a by-product of the reaction, 5-(diphenylphosphoryl)pentanoic acid. The suspension was stirred for 30 minutes at about 5° C. before filtration. The filter cake was rinsed with cold (about 5° C.) methyl t-butyl ether (30 mL). The filtrate was separated and the organic phase was washed with 10% brine (2×20 mL). To the organic phase was charged water (20 mL) and 1-adamantanamine (2.7 g, 17.8 mmol). The mixture was heated to reflux (about 56° C.) and maintained for 30 minutes prior to cooling to about 25° C. The suspension was stirred at about 25° C. overnight and filtered. The filter cake was rinsed with water (20 mL) and methyl t-butyl ether (40 mL). The damp cake was dried in vacuo (38 Torr) at about 45° C. to yield salt 6a as an off-white solid (6.16 g, 87.6% yield, HPLC Purity 94.39%).

### Example 14: Purification of Form APO-I of the Salt of Formula 6a

Salt 6a (2 g, 3.40 mmol), prepared as in Example 13, was charged to a mixture of water (20 mL) and acetone (2 mL). The mixture was heated to 60° C. and maintained for 30 minutes before it was allowed to cool to about 25° C. The mixture was stirred for a further 2 hours. The suspension was filtered and the filter cake was rinsed with a 10/1 (by volume) mixture of water/acetone (10 mL). Drying of the filter cake in vacuo (38 Torr) at about 45° C. gave salt 6a as an off-white solid (1.83 g, 91.5% yield, HPLC Purity 99.14%).

### Example 15: Purification of Form APO-I of the Salt of Formula 6a

Salt 6a (2 g, 3.40 mmol), prepared as in Example 13, was charged to a mixture of methyl t-butyl ether (20 mL) and acetone (2 mL). The mixture was heated to 60° C. and maintained for 30 minutes before cooling to about 25° C. The mixture was stirred for a further 2 hours. The suspension was filtered and the filter cake was rinsed with a 10/1 (by volume) mixture of methyl t-butyl ether/acetone (10 mL). Drying of the filter cake in vacuo (38 Torr) at about 45° C. gave salt 6a as an off-white solid (1.86 g, 93.0% yield, HPLC Purity 98.92%). A PXRD diffractogram of a sample prepared by this method is shown in FIG. 6.

### Example 16: Preparation of Tafluprost

A mixture of the compound of Formula 6a (10 g, 17.0 mmol), methyl t-butyl ether (100 mL) and i-propyl iodide (14.66 g, 86.3 mmol) was heated to reflux (about 56° C.). To the slurry was charged 1,8-diazabicycloundec-7-ene (15.76 g, 103.5 mmol) dropwise. Upon reaction completion (by thin-layer chromatography), the mixture was cooled to about 25° C., and then water (40 mL) was added. The organic phase was separated and washed with water (2×30 mL) and 1N aqueous HCl (30 mL). Concentration of the organic phase in vacuo (38 Torr, below 35° C.) yielded crude Tafluprost, which was purified by column chromatography on silica gel (loading: about 10 g silica (200-300 seive)/1 g crude) using methyl t-butyl ether/heptane (1/1) as eluent to afford purified Tafluprost (7.25 g, 94.3% yield, chromatographic purity: 99.38%)

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. Any priority document(s) are incorporated herein by reference as if each individual priority document were specifically and individually indicated to be incorporated by reference herein and as though fully set forth herein. The invention includes all embodiments and variations substantially as hereinbefore described and with reference to the examples and drawings.

1. A crystalline salt of a prostaglandin analog intermediate having a formula selected from the group consisting of:

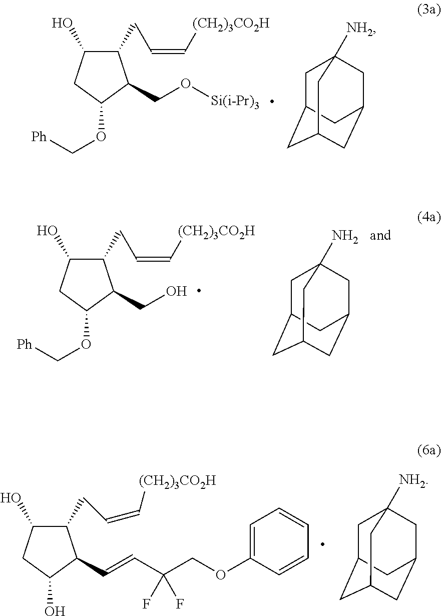

2. The crystalline salt of claim 1 having the Formula 3a.

3. The crystalline salt of claim 2, wherein the salt is characterized by a Powder X-Ray Diffraction (PXRD) diffractogram comprising a peak, expressed in degrees two-theta, at 11.0+/−0.2, and at least four peaks, expressed in degrees two-theta, selected from the group consisting of: 3.7+/−0.2, 7.5+/−0.2, 8.1+/−0.2, 9.5+/−0.2, 12.9+/−0.2, 13.6+/−0.2, 15.1+/−0.2, 15.7+/−0.2, 17.0+/−0.2, and 20.5+/−0.2.

4. The crystalline salt of claim 3, wherein the PXRD diffractogram comprises peaks, expressed in degrees two-theta, at: 3.7+/−0.2, 7.5+/−0.2, 8.1+/−0.2, 9.5+/−0.2, 11.0+/−0.2, 12.9+/−0.2, 13.6+/−0.2, 15.1+/−0.2, 15.7+/−0.2, 17.0+/−0.2 and 20.5+/−0.2.

5. The crystalline salt of claim 3, wherein the salt is characterized by a Differential Scanning Calorimetry (DSC) thermogram comprising an endothermic peak having a peak onset at approximately 78° C.

6. The crystalline salt of claim 1 having the Formula 4a.

7. The crystalline salt of claim 6, wherein the salt is Form APO-I characterized by a Powder X-Ray Diffraction (PXRD) diffractogram comprising a peak, expressed in degrees two-theta, at approximately 5.6+/−0.2, and at least four peaks, expressed in degrees two-theta, selected from the group consisting of: 7.5+/−0.2, 11.2+/−0.2, 15.2+/−0.2, 15.9+/−0.2, 16.9+/−0.2, 18.3+/−0.2, 18.9+/−0.2, 19.4+/−0.2, 21.2+/−0.2 and 23.7+/−0.2.

8. The crystalline salt of claim 7, wherein the PXRD diffractogram comprises peaks, expressed in degrees two-theta, at: 5.6+/−0.2, 7.5+/−0.2, 11.2+/−0.2, 15.2+/−0.2, 15.9+/−0.2, 16.9+/−0.2, 18.3+/−0.2, 18.9+/−0.2, 19.4+/−0.2, 21.2+/−0.2 and 23.7+/−0.2.

9. The crystalline salt of claim 7, wherein the salt is characterized by a Differential Scanning Calorimetry (DSC) thermogram comprising an endothermic peak with a peak onset at approximately 117° C.

10. The crystalline salt of claim 6 having a water content of less than about 0.5 wt %.

11. The crystalline salt of claim 6, wherein the salt is Form APO-II characterized by a Powder X-Ray Diffraction (PXRD) diffractogram comprising a peak, expressed in degrees two-theta, at approximately 14.2+/−0.2, and at least four peaks, expressed in degrees two-theta, selected from the group consisting of: 7.8+/−0.2, 10.0+/−0.2, 13.6+/−0.2, 15.5+/−0.2, 17.1+/−0.2, 17.9+/−0.2, 18.4+/−0.2, 19.5+/−0.2, 20.0+/−0.2, and 22.2+/−0.2.

12. The crystalline salt of claim 11, wherein the PXRD diffractogram comprises peaks, expressed in degrees two-theta, at: 7.8+/−0.2, 10.0+/−0.2, 13.6+/−0.2, 14.2+/−0.2, 15.5+/−0.2, 17.1+/−0.2, 17.9+/−0.2, 18.4+/−0.2, 19.5+/−0.2, 20.0+/−0.2, and 22.2+/−0.2.

13. The crystalline salt of claim 6, wherein the molar ratio of water to the salt is 0.5 to 1.

14. The crystalline salt of claim 1 having the Formula 6a.

15. The crystalline salt of claim 14, wherein the salt is characterized by a Powder X-Ray Diffraction (PXRD) diffractogram comprising a peak, expressed in degrees two-theta, at approximately 6.0+/−0.2, and at least four peaks, expressed in degrees two-theta, selected from the group consisting of 10.5+/−0.2, 12.1+/−0.2, 14.4+/−0.2, 16.0+/−0.2, 17.4+/−0.2, 18.3+/−0.2, 18.7+/−0.2, 19.4+/−0.2, 20.8+/−0.2, and 21.7+/−0.2.

16. The crystalline salt of claim 15, wherein the PXRD diffractogram comprises peaks, expressed in degrees two-theta, at: 6.0+/−0.2, 10.5+/−0.2, 12.1+/−0.2, 14.4+/−0.2, 16.0+/−0.2, 17.4+/−0.2, 18.3+/−0.2, 18.7+/−0.2, 19.4+/−0.2, 20.8+/−0.2, and 21.7+/−0.2.

17. The crystalline salt of claim 14, wherein the molar ratio of water to the salt is 1.5 to 1.

18. A process for the preparation of a salt of formula 6a: 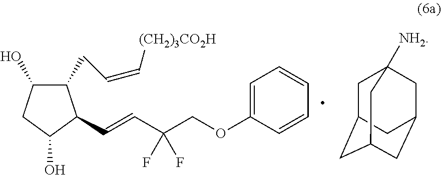
the process comprising:
i) combining 1-adamantanamine, water, an organic solvent selected from the group consisting of ethers, esters, ketones and aromatic hydrocarbons and a compound of Formula 6: 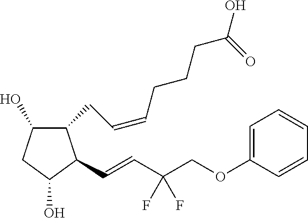 [SMILES: O=C(O)CCC/C=C\C[C@@H]1[C@@H](/C=C/C(F)(F)COc2ccccc2)[C@H](O)C[C@@H]1O]
thereby obtaining a suspension; and
ii) isolating the solid salt of Formula 6a from the suspension.

19. The process of claim 18, wherein the suspension is maintained for at least 30 minutes at a temperature of at least 40° C. prior to isolating the solid salt of Formula 6a from the suspension.

20. The process of claim 19, wherein isolating comprises drying in vacuo at a temperature below 50° C.

21. A process for the preparation of a salt of Formula 3a: 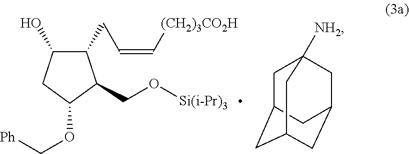
the process comprising:
a) combining, in a hydrocarbon solvent selected from the group consisting of heptane, pentane and cyclohexane, 1-adamantanamine and a compound of Formula 3: 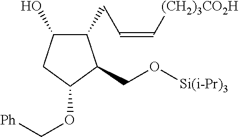
thereby obtaining a suspension; and
b) isolating the solid salt of Formula 3a from the suspension.
